(19) United States
(12) Patent Application Publication
(10) Pub. No.: US 2010/0249698 A1
(43) Pub. Date: Sep. 30, 2010

(54) CONTROLLED EXOTHERM OF CYANOACRYLATE FORMULATIONS

(76) Inventors: Binoy K. Bordoloi, Bridgewater, NJ (US); Melanie A. Vander Klok, Raleigh, NC (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/415,303

(22) Filed: Mar. 31, 2009

(51) Int. Cl. A61B 17/03; C09J 4/04

(52) U.S. Cl. 604/82; 523/118; 606/214

(57) ABSTRACT

The present invention is directed to curable cyanoacrylate-based formulation containing a multi-functional amine initiator. A dual function amine, particularly Quadrol, provides a highly desirable balance of maximum cure temperature and setting time of cure with the same higher viscosity, non-running cyanoacrylate formulation. The present invention is also directed to systems that utilize the inventive formulations and methods for use of such formulations.

### FIELD OF THE INVENTION

The invention relates to stabilized monomer and absorbable polymer adhesive and sealant compositions, and to their use for industrial and medical applications.

### BACKGROUND

Monomer and polymer adhesives/sealants are used in both industrial (including household) and medical/surgical applications. Included among these adhesives or sealants are cyanoacrylate monomers and polymers resulting therefrom. Since the discovery of the adhesive/sealant properties of such monomers and polymers, they have found wide use due to the speed with which they cure, the strength of the resulting bond formed, and their relative ease of use. These characteristics have made cyanoacrylate compositions the primary choice for numerous adhesive applications such as bonding plastics, rubbers, glass, metals, wood, and, more recently, medical, biological or living tissues.

Medical and surgical applications of cyanoacrylate compositions include their use as alternates or adjuncts to surgical sutures, meshes and staples or other medical devices in wound closure, as well as for covering and protecting surface wounds such as lacerations, abrasions, burns, stomatitis, sores, and other surface wounds. When a cyanoacrylate composition is applied, it is usually applied in its monomeric form, and the resultant polymer creates the desired adhesive bond or sealant strength.

During the cyanoacrylate adhesive polymerization process, an exothermic reaction occurs that increases the temperature of the composition. Depending on the monomer utilized in the composition and the additives used, the temperature increase varies. In particular, as the viscosity of the adhesive increases, the ability to apply a thicker layer of material in a single application is presented. This application creates the potential for a higher exotherm than if applying a less viscous composition using multiple layers to create an application of the same thickness. In addition, for some applications, it is desirable to use a more viscous adhesive in order to prevent the adhesive from running when applied to a surface and spreading into a wound or along a surface to an area that does not require adhesive.

The increase in temperature of the adhesive composition due to exothermic polymerization of the monomeric component may be as low as 5° C. and as high as 70° C., depending on the composition of the adhesive. A temperature increase of as little as 45° C. of the adhesive composition placed on the surface of living tissue will generally cause discomfort. It is widely believed that temperatures above 60° C. generally cause tissue damage.

As disclosed in U.S. Pat. No. 6,010,714 (the “714 patent”), which is hereby incorporated by reference herein, it is known to add a heat dissipating agent to a cyanoacrylate adhesive in order to reduce the amount of heat generated upon polymerization of the monomer. The '714 patent discloses adding heat dissipating agents such as ethers, ketones, chlorofluorocarbons, alkanes, alcohols, alkenes and mixtures thereof. The heat dissipating agents disclosed in the '714 patent are useful for adhesive compositions having relatively low viscosities, e.g. 40-50 cp. There remains a need for a relatively thicker surgical adhesive that does not cause thermal damage or necrosis of living tissue after application of the adhesive to the tissue. Therefore, there is a need for an additive or a combination of additives that will reduce the amount of heat released or exotherm generated during the polymerization of a cyanoacrylate monomer.

Other background information is available in McMahon, R., M. Brennan, and J. D. Glennon, “The pKa values of N,N,N′,N′-(2-hydroxypropyl)ethylenediamine”; Talanta, 33(11), 927 (1986); and J G Woods et al, “Multi-amine Compound Primers for Bonding of Polyolefins with Cyanoacrylate Adhesives”, U.S. Pat. No. 6,673,192 B1 (January 2004).

### SUMMARY

An adhesive composition is provided comprising one or more polymerizable cyanoacrylate monomers, a multi-functional polymerization initiator having at least two amine moieties having different pKa values in the same molecule for the one or more polymerizable cyanoacrylate monomers. The adhesive composition may further comprise one or more of stabilizing agents, preservatives, heat dissipating agents, colorant, or combinations thereof.

The preferred initiators are dual functional polymerization initiators described herein are N,N,N′,N′-tetrakis(2-hydroxypropyl)ethylenediamine (referred to herein as TKHPED), available under the tradename Quadrol from BASF and an analogous compound N,N,N′,N′-tetrakis(2-hydroxyethyl)ethylene diamine (referred to herein as TKHEED, available from TCI America, Inc.), generally represented by the following formula:

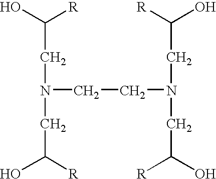

where each R group is independently H, C₁-C₃ alkyl.

An example of a dual functional polymerization initiator (When R═CH₃) is represented by the following formula A:

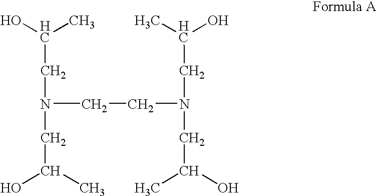

In an embodiment, a system for treating living tissue is provided comprising a first reservoir containing one or more polymerizable cyanoacrylate monomers, a second reservoir in a non-contacting relationship with the first reservoir containing a dual functional polymerization initiator for the one or more polymerizable cyanoacrylate monomers, and an applicator capable of combining the polymerizable cyanoacrylate monomer and the dual functional polymerization initiator to form an adhesive composition and then applying the adhesive composition to living tissue. The dual functional polymerization initiator for the one or more polymerizable cyanoacrylate monomers comprises two amine moieties having two distinct pKa values.

In another embodiment, a method of treating living tissue is provided comprising applying to living tissue a biocompatible adhesive composition comprising one or more polymerizable cyanoacrylate monomers, a dual functional polymerization initiator for the one or more polymerizable cyanoacrylate monomers, comprising two amine moieties of different pKa values.

### DETAILED DESCRIPTION

For the purposes of this invention, the term “absorbable” or variations thereof means capable of being absorbed, degraded or biodegraded, either fully or partially, by animal (including human) tissue after application of the adhesive or sealant. Also, the term “substantially absorbed” means at least 90% absorbed. The term “non-absorbable” or variations thereof means completely or substantially incapable of being absorbed, either fully or partially, by animal tissue after application of the adhesive or sealant.

The term “effective amount” is an amount sufficient to provide desired properties to the adhesive compositions. The effective amount may be affected by cyanoacrylate monomers, viscosity modifying agents, stabilizers, initiators or other ingredients used to form the adhesive composition.

The term “stability” or “stabilized” as used herein may be determined by measuring the viscosity of the cyanoacrylate composition over a period of time. Premature polymerization of the cyanoacrylate composition results in an increase in viscosity over time; therefore, viscosity of a composition may be used to determine composition stability.

The term “biocompatible” refers to a material being suited for and meeting the requirements of a medical device, used for either long or short term implants or for non-implantable applications, such that when implanted or applied in an intended location, the material serves the intended function for the required amount of time without causing an unacceptable response. Long term implants are defined as items implanted for more than 30 days.

The term “multi-functional amine” refers to a compound having at least two amine moieties with each moiety having a different pKa value. An example of a dual functional amine is Quadrol, a compound with pKa values of 4.3 and 8.99, respectively, for the quaternary and tertiary amine groups therein. The proton dissociation constants, pKa, of Quadrol is determined by potentiometric titration at 25° C. in 0.15 M sodium chloride solution. Thus one of the amine groups would exist in a cationic or a quaternary ammonium form (pKa₂=4.30±0.04) at pH of 7, while the other would be neutral or uncharged tertiary form (pKa₁=8.99±0.04). At a pH of greater than 8.99, both amines would be in uncharged form.

A cyanoacrylate adhesive composition comprising one or more polymerizable cyanoacrylate monomer is provided. Controlled exotherm of the monomeric cyanoacrylate adhesive composition is achieved through the addition of a multi-functional initiator comprising two amine moieties of different pKa values to a polymerizable monomeric cyanoacrylate composition resulting in a cyanoacrylate adhesive composition. The multi-functional initiator composition enables the reduction of exotherm upon polymerization of the cyanoacrylate monomer without any detrimental effect on the speed of curing.

A suitable dual functional amine may serve as the initiator and may have a cure rate of as short a time as a few seconds to a few minutes. The cure rate may be closely controlled by the selection of an amount or concentration of the dual functional amine to be added to the composition and may thus be readily controlled by one skilled in the art in light of the present disclosure. A suitable dual functional amine provides consistent, controllable, and complete polymerization of the monomer or monomers so that the polymerization of the monomer or monomers can be made to occur in the time desired for the particular application.

The amount of the dual functional amine initiator added to the polymerizable cyanoacrylate monomer typically may depend on the cyanoacrylate monomer(s), the accelerator, the viscosity modifying agent, the stabilizers, and the desired rate of polymerization. Typically, the dual functional amine initiator will be present in an amount of from about 10 ppm to about 10,000 ppm. When applied to the medical device, the concentrations will be present in an amount of from about 500 to 3000 microgram.

The effectiveness of the dual functional amine initiator may be demonstrated by comparison made with a blend of two amines, one a quaternary ammonium salts, for example, benzalkonium chloride (BAC) and the other a tertiary amine for example, triisopropanol amine (TIPA), where the former may act as an initiator and latter an accelerator. Similarly, the dual functional amine initiator provides consistent, controllable, and complete polymerization of the monomer or monomers so that the polymerization of the monomer or monomers can be made to occur in the time desired for the particular application. In addition, the dual functional amine initiator reduces the amount of heat released upon polymerization of the cyanoacrylate monomer.

Cyanoacrylate adhesive monomer compositions including the multi-functional amine initiator initiators as described, and polymers formed therefrom, are useful as tissue adhesives, sealants for preventing bleeding or for covering open wounds, and in other biomedical applications. The adhesive compositions find uses in, for example, preventing body fluid leakage, sealing air leakage in the body, tissue approximation, apposing surgically incised or traumatically lacerated tissues; retarding blood flow from wounds; drug delivery; dressing burns; dressing skin or other superficial or deep tissue surface wounds (such as abrasions, chaffed or raw skin, and/or stomatitis); and aiding repair and re-growth of living tissue. Adhesive compositions of the present invention have broad application for sealing wounds in various living tissue, internal organs and blood vessels, and can be applied, for example, on the interior or exterior of blood vessels and various organs or tissues. Adhesive compositions of the present invention are also useful as antimicrobial barriers. Adhesive compositions of the present invention are also useful in industrial and home applications, for example in bonding rubbers, plastics, wood, composites, fabrics, and other natural and synthetic materials.

Monomers that may be used in this invention are readily polymerizable, e.g. anionically polymerizable or free radical polymerizable, or polymerizable by zwitterions or ion pairs to form polymers. Some such monomers are disclosed in, for example, U.S. Pat. No. 5,328,687 to Leung, et al., which is hereby incorporated by reference in its entirety herein. Preferably, the cyanoacrylate adhesive compositions comprise one or more polymerizable cyanoacrylate monomers and are biocompatible. The cyanoacrylate adhesive compositions comprising one or more polymerizable cyanoacrylate monomers may include combinations or mixtures of cyanoacrylate monomers.

Preferably, the adhesive composition comprises one or more polymerizable cyanoacrylate monomers and may include combinations or mixtures of cyanoacrylate monomers of formula (I). The cyanoacrylates monomers have the formula

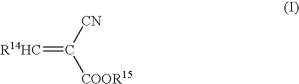

wherein R¹⁴ is hydrogen and R¹⁵ is a hydrocarbyl or substituted hydrocarbyl group; a group having the formula —R¹⁶—O—R¹⁷—O—R¹⁸, wherein R¹⁶ is a 1,2-alkylene group having 2-4 carbon atoms, R¹⁷ is an alkylene group having 1-4 carbon atoms, and R¹⁸ is an alkyl group having 1-6 carbon atoms; or a group having the formula

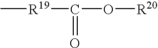

wherein R¹⁹ is

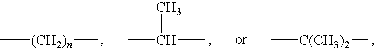

wherein n is 1-10, preferably 1-5 carbon atoms, and R²⁰ is an organic moiety. The organic moiety R²⁰ may be substituted or unsubstituted and may be straight chain, branched or cyclic, saturated, unsaturated or aromatic. Preferred organic radicals are alkyl, alkenyl, and alkynyl moieties having from 1 to about 8 carbon atoms, and halo-substituted derivatives thereof. Particularly preferred are alkyl moieties of 4 to 6 carbon atoms.

In the cyanoacrylate monomer of formula (I), R¹⁵ may be an alkyl group having 1 -10 carbon atoms or a group having the formula —AOR²¹, wherein A is adivalent straight or branched chain alkylene or oxyalkylene moiety having 2-8 carbon atoms, and R²¹ is a straight or branched alkyl moiety having 1-8 carbon atoms. Examples of groups represented by the formula —AOR²¹ include 1-methoxy-2-propyl, 2-butoxy ethyl, isopropoxy ethyl, 2-methoxy ethyl, and 2-ethoxy ethyl.

The cyanoacrylates of formula (I) can be prepared according to methods known in the art. For example, cyanoacrylates can be prepared by reacting an alkyl cyanoacetate with formaldehyde in a nonaqueous organic solvent and in the presence of a basic catalyst, followed by pyrolysis of the anhydrous intermediate polymer in the presence of a polymerization inhibitor as disclosed in U.S. Pat. Nos. 2,721,858 and 3,254,111. The cyanoacrylates of formula (I) wherein R¹⁵ is a group having the formula R¹⁶—O—R¹⁷—O—R¹⁸ can be prepared according to the method disclosed in U.S. Pat. No. 4,364,876, and the cyanoacrylates of formula (I) wherein R¹⁵ is a group having the formula

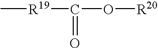

can be prepared according to the method described in U.S. Pat. No. 3,995,641. Each of the above listed patents is hereby incorporated by reference in its entirety.

Suitable cyanoacrylate monomers may be used, alone or in combination, and may include, but not be limited to, 2-octyl cyanoacrylate; dodecyl cyanoacrylate; 2-ethylhexyl cyanoacrylate; butyl cyanoacrylate such as n-butyl cyanoacrylate; ethyl cyanoacrylate; methyl cyanoacrylate; methoxyethyl cyanoacrylate; 2-ethoxyethyl cyanoacrylate; 3-methoxybutyl cyanoacrylate; 2-butoxyethyl cyanoacrylate; 2-isopropoxyethyl cyanoacrylate; and 1-methoxy-2-propyl cyanoacrylate. In embodiments, the monomers may be ethyl, n-butyl, or 2-octyl α-cyanoacrylate.

The cyanoacrylate monomers which may be used in the adhesive/sealant as biocompatible compositions may include alkyl ester cyanoacrylates. The alkyl ester cyanoacrylate monomers may have the formula:

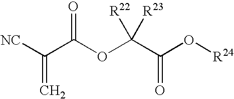

wherein R²² and R²³ are, independently, H, a straight, branched or cyclic alkyl, or are combined together in a cyclic alkyl group, R²⁴ is a straight, branched or cyclic alkyl group., Preferably, R²² is H or a C₁, C₂ or C₃ alkyl group, such as methyl or ethyl; R²³ is H or a C₁, C₂ or C₃ alkyl group, such as methyl or ethyl; R²⁴ is a C₁-C₁₆ alkyl group, more preferably a C₁-C₁₀ alkyl group, such as methyl, ethyl, propyl, isopropyl, butyl, isobutyl, pentyl, hexyl, heptyl, octyl, nonyl or decyl, and even more preferably a C₂, C₃ or C₄ alkyl group.

Examples of alkyl ester cyanoacrylates include, but are not limited to, butyl lactoyl cyanoacrylate (BLCA), butyl glycoloyl cyanoacrylate (BGCA), isopropyl glycoloyl cyanoacrylate (IPGCA), ethyl lactoyl cyanoacrylate (ELCA), and ethyl glycoloyl cyanoacrylate (EGCA), isopropylethylcyanoacrylate (IPECA) and combinations thereof. BLCA may be represented by the formula above, wherein R²² is H, R²³ is methyl and R²⁴ is butyl. BGCA may be represented by the formula above, wherein R²² is H, R²³ is H and R²⁴ is butyl. IPGCA may be represented by the formula above, wherein R²² is H, R²³ is H and R²⁴ is isopropyl. ELCA may be represented by the formula above, wherein R²² is H, R²³ is methyl and R²⁴ is ethyl. EGCA may be represented by the formula above, wherein R²² is H, R²³ is H and R²⁴ is ethyl.

Other examples of alkyl ester cyanoacrylates include 3-(2-Cyano-acryloyloxy)-butyric acid ethyl ester (Et-β-HBT-CA), 3-(2-cyano-acryloyloxy)-hexanoic acid ethyl ester (Et-β-CPL-CA), alkyl alpha-cyanoacryloyl caprolactate and alkyl alpha-cyanoacryloyl butrylactate.

The alkyl ester cyanoacrylate monomers may be prepared through the Knoevenagel reaction of an alkyl cyanoacetate, or an alkyl ester cyanoacetate, with paraformaldehyde as disclosed in U.S. Pat. No. 3,995,641. This leads to a cyanoacrylate oligomer. Subsequent thermal cracking of the oligomer results in the formation of a cyanoacrylate monomer. After further distillation, a cyanoacrylate monomer with high purity (greater than 95.0%, preferably greater than 99.0%, and more preferably greater than 99.8%) may be obtained. Monomers prepared with low moisture content and essentially free of impurities (e.g., surgical grade) are preferred for biomedical use.

An alternative or additional cyanoacrylate which may be used in the adhesive/sealant compositions includes alkyl ether cyanoacrylate. Alkyl ethyl cyanoacrylates have the general formula:

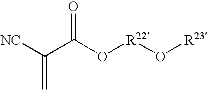

wherein R^{22′} is a straight, branched or cyclic alkyl, and R^{23′} is a straight, branched or cyclic alkyl group. Preferably, R^{22′} is a C₁, C₂ or C₃ alkyl group, such as methyl or ethyl; and R^{23′} is a C₁-C₁₆ alkyl group, more preferably a C₁-C₁₀ alkyl group, such as methyl, ethyl, propyl, isopropyl, butyl, isobutyl, pentyl, hexyl, heptyl, octyl, nonyl or decyl, and even more preferably a C₂, C₃ or C₄ alkyl group.

Examples of alkyl ether cyanoacrylates include, but are not limited to, isopropyoxy ethyl cyanoacrylate (IPECA) and methoxy butyl cyanoacrylate (MBCA) or combinations thereof. IPECA may be represented by the formula above, wherein R^{22′} is ethylene and R^{23′} is isopropyl. MBCA may be represented by the formula above, wherein R^{22′} is n-butylene and R^{23′} is methyl.

Additional examples of suitable cyanoacrylates for internal application are α-cyanoacrylate monomers described in U.S. Pat. No. 7,238,828, which is incorporated herein by reference.

The α-cyanoacrylate monomers are alkyl ester α-cyanoacrylate monomers of the general formula having a spacer R1:

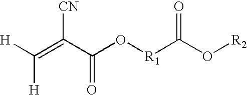

wherein

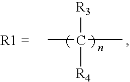

wherein n is from 2 to 12; R3 and R4 is an alkyl group or a hydrogen, and at least one of R3 or R4 is an alkyl group (e.g. linear or branched, or cyclic) having from 1, 2, 3, 4, 5, 6, 7, 8, 9, 10, 11, 12 and 13 carbon atoms; R2 is an alkyl group (e.g. linear or branched, or cyclic) having from 1, 2, 3, 4, 5, 6, 7, 8, 9, 10, 11, 12 and 13 carbon atoms; and the combined number of carbon atoms (N) in the spacer R1 is at least n+1.

Alkyl ester cyanoacrylates and alkyl ether cyanoacrylates are particularly useful for medical applications and internal use because of their absorbability by living tissue and associated fluids. It is desirable that 100% of the polymerized and applied cyanoacrylate adhesive be absorbed in a period of less than 3 years, preferably approximately 1-24 months, more preferably 1-18 months, and most preferably 3-12 months after application of the adhesive to living tissue. The absorption time may vary depending on the particular uses and tissues involved. It may be desirable for the absorption time to be longer for some types of tissue and to be shorter for other tissue types. For example, a longer absorption time may be desired when the adhesive composition is applied to hard tissues, such as bone, but a shorter absorption time may be desired when the adhesive composition is applied to softer tissues.

The selection of monomer will affect the absorption rate of the resultant polymer, as well as the polymerization rate of the monomer. Thus, two or more different monomers having varied absorption and/or polymerization rates may be used in combination to give a greater degree of control over the absorption rate of the resultant polymer, as well as the polymerization rate of the monomer. The adhesive composition may comprise a mixture of monomer species with varying absorption rates. Where two monomer species having different absorption rates are used, it is preferred that the absorption rates be sufficiently different that a mixture of the two monomers can yield a third absorption rate that is effectively different from the absorption rates of the two monomers individually. Compositions according to these embodiments are described, for example, in U.S. Patent Publication No. 2002/0037310 and U.S. Pat. No. 6,620,846, both incorporated herein by reference in their entireties.

Suitable monomer compositions may be prepared by mixing suitable quantities of an alkyl α-cyanoacrylate such as 2-octyl α-cyanoacrylate with one of butyl lactoyl cyanoacrylate (BLCA), butyl glycoloyl cyanoacrylate (BGCA), isopropyl glycoloyl cyanoacrylate (IPGCA), ethyl lactoyl cyanoacrylate (ELCA), and ethyl glycoloyl cyanoacrylate (EGCA). Such mixtures may range from ratios of about 90:10 to about 10:90 by weight, preferably about 75:25 to about 25:75 by weight.

A stabilizer or stabilizing agent may be added to the composition to prevent premature polymerization or to increase the shelf life of the cyanoacrylate monomeric composition. For example, boron trifluoride may be used as a stabilizing agent. Other suitable free radical stabilizing agents for use in monomeric cyanoacrylate compositions include, but are not limited to, hydroquinone, hydroquinone monomethyl ether, catechol, pyrogallol, benzoquinone, 2-hydroxybenzoquinone, p-methoxy phenol, t-butyl catechol, butylated hydroxy anisole, butylated hydroxy toluene, and t-butyl hydroquinone and mixtures or combinations thereof. The free radical stabilizing agents may be used in amounts from about 5 to about 10,000 ppm. In exemplary embodiments, if hydroquinone is used, the amount may be from about 5 to about 2000 ppm and may be used in conjunction with butylated hydroxy anisole in an amount of about 500 to about 10,000 ppm.

The cyanoacrylate adhesive compositions may also optionally include at least one anionic vapor phase stabilizer and at least one anionic liquid phase stabilizer. Examples of such anionic agents are described for example, in U.S. Pat. No. 6,620,846, incorporated herein by reference in its entirety.

The anionic vapor phase stabilizers may be selected from among known stabilizers, including, but not limited to, sulfur dioxide, boron trifluoride, or hydrogen fluoride. Typically, each anionic vapor phase stabilizer is added in such an amount to give a concentration of less than about 200 parts per million (ppm). In exemplary embodiments, each anionic vapor phase stabilizer is present in an amount from about 1 to about 200 ppm, preferably from about 3 to about 75 ppm, even more preferably from about 3 to about 50 ppm, and most preferably from about 3 to about 20 ppm.

The liquid phase anionic stabilizer is a very strong acid that has an aqueous pKₐ of less than 1.0. Examples of such very strong acids include, but are not limited to, sulfuric acid (pKₐ−3.0), perchloric acid (pKₐ−5), hydrochloric acid (pKₐ−7.0), hydrobromic acid (pKₐ−9), fluorosulfonic acid (pKa<−10), and chlorosulfonic acid (pKₐ−10). In embodiments, the very strong acid liquid phase anionic stabilizer is added in an amount to give a final concentration of about 1 to about 200 ppm. The very strong acid liquid phase anionic stabilizer may be present in a concentration of from about 5 to about 80 ppm, preferably from about 5 to about 40 ppm. For example, the very strong acid liquid phase anionic stabilizer may be sulfuric acid or chlorosulfonic acid.

The adhesive composition may optionally include at least one secondary anionic active agent. The secondary anionic active agents may be included in the adhesive compositions to more precisely control the cure speed and stability of the adhesive as well as the molecular weight of the cured adhesive. The secondary anionic active agent would typically be an acid with a higher pKₐ ranging from 2 to 8, preferably from 2 to 6, and most preferably from 2 to 5. Examples of such suitable secondary anionic active agents include, but are not limited to, phosphoric acid (pKₐ 2.2), organic acids, such as acetic acid (pKₐ 4.8), benzoic acid (pKₐ 4.2), chloroacetic acid (pKₐ 2.9), cyanoacetic acid, and mixtures thereof. For example, an amount of acetic acid and/or benzoic acid may be about 25 to about 500 ppm. For acetic acid, the concentration may typically be about 50 to about 400 ppm, preferably about 75 to about 300 ppm, and more preferably about 100 to about 200 ppm, which is described in U.S. Pat. No. 5,981,621 incorporated herein by reference.

Any mixture of stabilizers and/or secondary anionic active agents may be included in the adhesive composition as long as the mixture does not significantly inhibit the desired polymerization rate of the composition. It is generally desirable for the polymerization rate of a composition to be in a range of about thirty seconds to about five minutes. Therefore, a mixture of stabilizers and/or secondary anionic active agents that inhibit polymerization such that the polymerization rate is outside of the preferred rate window may be undesirable. Furthermore, the mixture should not, in medical adhesive compositions, show unacceptable levels of toxicity. One of ordinary skill in the art will know the levels of toxicity that are acceptable for medical uses. Thus, the amount of stabilizers and/or anionic active agents to be used can be determined by one of ordinary skill in the art without undue experimentation.

The stabilizers and secondary anionic active agents are chosen such that they are compatible with the chosen adhesive composition including the cyanoacrylate monomers, boron trifluoride and other stabilizers, as well as with the packaging material and the equipment used to make and package the composition. Hence, a suitable combination should be a viscous, stabilized and substantially unpolymerized adhesive composition after packaging and sterilization.

The addition of these stabilizing agents to the cyanoacrylate monomer compositions may affect cure or polymerization rate of the compositions. To overcome the slow polymerization, a compatible agent which promotes initiation or acceleration of polymerization of a cyanoacrylate monomer or a mixture of cyanoacrylate monomers may be used with the monomer composition. For some medical applications, initiators or rate modifying agents providing a faster cure rate while maintaining the absorbability of the monomer composition are preferred.

In exemplary embodiments, the dual functional polymerization initiators described herein are N,N,N′,N′- tetrakis(2-hydroxypropyl)ethylenediamine (referred to herein as TKHPED), available under the tradename Quadrol from BASF and an analogous compound N,N,N′,N′-tetrakis(2-hydroxyethyl)ethylene diamine (referred to herein as TKHEED, available from TCI America, Inc.), generally represented by the following formula:

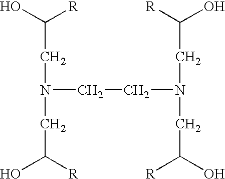

where each R group is independently H, C₁-C₃ alkyl.

An example of a dual functional polymerization initiator (When R═CH₃) is represented by the following formula. The dual functional initiator with one amine moiety in a quaternary ammonium form can have hydroxyl as the counter anion.

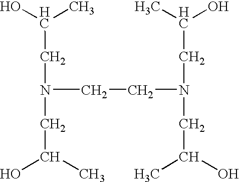

Mixtures of two or more initiators or rate modifying agents may be used with at least one dual functional polymerization diamine initiators and a second initiator. A combination of multiple initiators or rate modifying agents may be beneficial in order to tailor the initiation of the polymerizable monomer species. For example, when a mixture of monomers is used, a mixture of initiators may provide superior results in comparison to a single initiator. Also a mixture of initiators may provide one initiator that preferentially initiates one monomer, and a second initiator that preferentially initiates the other monomer, or may provide initiation rates to help ensure that both monomer species are initiated at equivalent, or desired non-equivalent, rates. In this manner, a mixture of initiators may advantageously minimize the amount of initiator to be used. Furthermore, a mixture of initiators may advantageously enhance the polymerization reaction kinetics.

The second initiator may include quaternary ammonium chloride and bromide salts. By way of example, quaternary ammonium salts such as domiphen bromide, butyrylcholine chloride, benzalkonium bromide, benzalkonium chloride, acetyl choline chloride, among others, may be used. When the benzalkonium halide is used, it may be benzalkonium halide in its unpurified state, which comprises a mixture of varying chain-length compounds, or it may be any suitable purified compound including those having a chain length of from about 12 to about 18 carbon atoms, including but not limited to C₁₂, C₁₃, C₁₄, C₁₅, C₁₆, C₁₇, and C₁₈ compounds.

The second initiator or rate modifying agent may also be selected by one of ordinary skill in the art without undue experimentation. Such suitable initiators or rate modifying agents may include, but are not limited to, detergent compositions; surfactants: e.g., nonionic surfactants such as Polysorbate 20 (e.g., Tween 20™ from ICI Americas), Polysorbate 80 (e.g., Tween 80™ from ICI Americas) and poloxamers, cationic surfactants such as tetrabutylammonium bromide, anionic surfactants such as sodium tetradecyl sulfate, and amphoteric or zwitterionic surfactants such as dodecyldimethyl(3-sulfopropyl)ammonium hydroxide, inner salt; amines, imines and amides, such as imidazole, arginine and povidine; phosphines, phosphites and phosphonium salts, such as triphenylphosphine and triethyl phosphite; alcohols such as ethylene glycol, methyl gallate; tannins; inorganic bases and salts, such as sodium bisulfite, calcium sulfate and sodium silicate; sulfur compounds such as thiourea and polysulfides; polymeric cyclic ethers such as monensin, nonactin, crown ethers, calixarenes and polymeric-epoxides; cyclic and acyclic carbonates, such as diethyl carbonate; phase transfer catalysts such as Aliquat 336; organometallics such as cobalt naphthenate and manganese acetylacetonate; radical initiators or accelerators, such as di-t-butyl peroxide and azobisisobutyronitrile; and a catalytic amount of an amine activated free radical initiator, accelerator, or rate modifier.

The multi-functional polymerization amine initiators or rate modifying agent may be in the form of a solid, such as a powder or a solid film, or in the form of a liquid, such as a viscous or paste-like material. The dual functional polymerization amine initiator or rate modifying agent may also include a variety of additives, such as surfactants or emulsifiers. Preferably, the dual functional polymerization amine initiator is soluble in the monomer composition, and/or comprises or is accompanied by at least one surfactant which, in embodiments, helps the initiator or co-elute with the monomer composition. In embodiments, the surfactant may help disperse the initiator in the monomer composition.

The multi-functional polymerization amine initiator or rate modifying agent may be applied to the tissue or surface being treated before the monomer composition, or may be applied directly to the monomer composition when the composition is applied to the tissue. The multi-functional polymerization r or rate modifying agent, when present, may be combined with the monomer composition just prior to applying the composition to tissue.

The selection of a multi-functional polymerization amine or rate modifying agent, when used, may additionally affect the rate at which the polymerized monomer is absorbed by living tissue. Therefore, for some medical applications, the most suitable initiators or rate modifying agents are those that initiate or accelerate polymerization of the monomer at a rate suitable for medical applications while providing a polymer that is substantially absorbed in less than three years. For purposes herein, the phrase “suitable for medical application(s)” means that the polymerization of the monomer occurs in less than 5 minutes or less than 3 minutes, preferably in less than 2.5 minutes, more preferably in less than 1 minute, and often in less than 45 seconds. The desired polymerization time may vary for different compositions and/or applications.

Other optional components may be present in the polymerizable cyanoacrylate compositions including, but not limited to, preservatives, heat dissipating agents, plasticizers, viscosity modifying agents, thixotropic agents, and colorants, which are described herein. Typically, these components will be used in amounts of up to about 25 weight %, more preferably up to about 10 weight %, and most preferably, up to about 5 weight %, based on a total weight of the composition.

The preservative may be selected from among preservatives including, but not limited to, parabens and cresols. For example, suitable parabens include, but are not limited to, alkyl parabens and salts thereof, such as methylparaben, methylparaben sodium, ethylparaben, propylparaben, propylparaben sodium, butylparaben, and the like. Suitable cresols include, but are not limited to, cresol, chlorocresol, and the like. The preservative may also be selected from other known agents including, but not limited to, hydroquinone, pyrocatechol, resorcinol, 4-n-hexyl resorcinol, captan (i.e., 3a,4,7,7a- tetrahydro-2-((trichloromethyl)thio)-1H-isoindole-1,3(2H)-dione), benzoic acid, benzyl alcohol, chlorobutanol, dehydroacetic acid, o-phenylphenol, phenol, phenylethyl alcohol, potassium benzoate, potassium sorbate, sodium benzoate, sodium dehydroacetate, sodium propionate, sorbic acid, thimerosal, thymol, phenylmercuric compounds such as phenylmercuric borate, phenylmercuric nitrate and phenylmercuric acetate, formaldehyde, and formaldehyde generators such as the preservatives Germall II® and Germall 115® (imidazolidinyl urea, available from Sutton Laboratories, Charthan, N.J.). Other suitable preservatives are disclosed in U.S. Pat. No. 6,579,469, the entire disclosure of which is hereby incorporated by reference. In embodiments, mixtures of two or more preservatives may also be used.

The heat dissipating agent may include liquids or solids that may be soluble or insoluble in the monomer. The liquids may be volatile and may evaporate during polymerization, thereby releasing heat from the composition. Suitable heat dissipating agents may be found in U.S. Pat. No. 6,010,714, the entire disclosure of which is incorporated herein.

The plasticizing agent imparts flexibility to the polymer that is formed from the monomer. The plasticizing agent preferably contains little or no moisture and should not significantly affect the stability or polymerization of the monomer. Examples of suitable plasticizers include acetyl tributyl citrate, dimethyl sebacate, triethyl phosphate, tri(2-ethylhexyl)phosphate, tri(p-cresyl)phosphate, glyceryl triacetate, glyceryl tributyrate, dibutyl sebacate, di-n-butyl sebacate, diethyl sebacate, dioctyl adipate, isopropyl myristate, butyl stearate, lauric acid, trioctyl trimellitate, dioctyl glutarate, polydimethylsiloxane, and mixtures thereof. Preferred plasticizers include di-n-butyl sebacate. In embodiments, suitable plasticizers include polymeric plasticizers, such as polyethylene glycol (PEG) esters and capped PEG esters or ethers, polyester glutarates and polyester adipates.

The viscosity of the polymerizable cyanoacrylate monomer or monomers and/or the monomer composition may be controlled by the addition of a viscosity modifying agent or component. The viscosity modifying agents may be selected from among known thickeners, including, but not limited to, poly(2-ethylhexyl methacrylate), poly(2-ethylhexyl acrylate) and cellulose acetate butyrate. Suitable thickeners further include, for example, polycyanoacrylates, polyoxalates, lactic-glycolic acid copolymers, polycaprolactone, lactic acid-caprolactone copolymers, poly(caprolactone+DL-lactide+glycolide), polyorthoesters, polyalkyl acrylates, copolymers of alkylacrylate and vinyl acetate, polyalkyl methacrylates, and copolymers of alkyl methacrylates and butadiene. Examples of alkyl methacrylates and acrylates are poly(butylmethacrylate) and poly(butylacrylate), also copolymers of various acrylate and methacrylate monomers, such as poly(butylmethacrylate-co-methylmethacrylate). Biodegradable polymer thickeners are preferred for some uses such as some surgical uses.

Preferably, the viscosity modifying agent is soluble in a monomer composition at room temperature (i.e., 20-25° C.) so that it may be added to the monomer composition without excessive heating of the monomer composition and remain uniformly combined in the composition.

The amount of viscosity modifying agent that is added to the monomer composition depends upon the molecular weight of the viscosity modifying agent. The viscosity modifying agent preferably comprises from about 0.5 to about 25.0% by weight of the adhesive composition. In preferred embodiments, the viscosity modifying agent comprises from about 1.0 to about 10.0%, more preferably about 1.0 to about 5.0%, of the adhesive composition. In embodiments, the viscosity modifying agent has a high molecular weight, preferably at least 100,000, or at least 500,000 or at least 1,000,000. The viscosity modifying agent is selected such that it is compatible with the monomer (i.e., does not adversely affect polymerization, bond strength, core properties, or shelf-life). The amount of viscosity modifying agent to be used can be determined by one of ordinary skill in the art using known techniques without undue experimentation.

In embodiments, the adhesive composition has a viscosity of about 20-10,000 centipoise, preferably 30-1,000 centipoise, and more preferably 200-1,000 centipoise as measured with a Brookfield Viscometer at 25° C.

Suitable thixotropic agents may include, but are not limited to, silica gels such as those treated with a silyl isocyanate. Examples of suitable thixotropic agents are disclosed in, for example, U.S. Pat. No. 4,720,513, the disclosure of which is hereby incorporated in its entirety.

The composition may also optionally include at least one natural or synthetic rubber to impart impact resistance, which is preferable especially for industrial compositions of the present invention. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties.

Compositions of the present invention are believed to have little to no toxicity. Nevertheless, medical compositions of the present invention may also include at least one biocompatible agent effective to reduce active formaldehyde concentration levels produced during in vivo biodegradation of the polymer (also referred to herein as “formaldehyde concentration reducing agents”). Preferably, this component is a formaldehyde scavenger compound. Examples of formaldehyde scavenger compounds include sulfites; bisulfites; mixtures of sulfites and bisulfites; ammonium sulfite salts; amines; amides; imides; nitriles; carbamates; alcohols; mercaptans; proteins; mixtures of amines, amides, and proteins; active methylene compounds such as cyclic ketones and compounds having a α-dicarbonyl group; and heterocyclic ring compounds free of a carbonyl group and containing an NH group, with the ring made up of nitrogen or carbon atoms, the ring being unsaturated or, when fused to a phenyl group, being unsaturated or saturated, and the NH group being bonded to a carbon or a nitrogen atom, which atom is directly bonded by a double bond to another carbon or nitrogen atom. Other examples of formaldehyde level reducing compounds and compositions are disclosed in exemplary patents U.S. Pat. Nos. 6,010,714; 5,624,669; 5,582,834; and 5,575,997, the entire disclosures of which are hereby incorporated by reference.

To improve the cohesive strength of adhesives formed from the compositions of this invention, difunctional monomeric cross-linking agents may be added to the monomer compositions of this invention. Such crosslinking agents are known. Exemplary crosslinking agents are disclosed in U.S. Pat. No. 3,940,362, which is hereby incorporated by reference in its entirety. Examples of suitable crosslinking agents include, but are not limited to, alkyl bis(2-cyanoacrylates), triallyl isocyanurates, alkylene diacrylates, alkylene dimethacrylates, trimethylol propane triacrylate, and alkyl bis(2-cyanoacrylates).

To improve the adhesion between substrates (e.g. tissue surface) and the compositions of this invention, priming agents may be used to condition the substrate prior to applying in the cyanoacrylate monomer. Suitable primers include, but are not limited to, pH-modifying agents (e.g. organic or inorganic bases), ionic and non-ionic surfactants, and organic or inorganic salts. Other suitable priming agents can be readily identified by one skilled in the art in light of the present disclosure.

The compositions of this invention may further contain fibrous reinforcements and colorants such as dyes, pigments, and pigment dyes. Examples of suitable fibrous reinforcements include PGA microfibrils, collagen microfibrils, cellulosic microfibrils, and olefinic microfibrils. Examples of suitable colorants include 1-hydroxy-4-[4-methylphenyl-amino]-9,10 anthracenedione (D+C violet No. 2); disodium salt of 6-hydroxy-5- [(4-sulfophenyl)axo]-2-naphthalene-sulfonic acid (FD+C Yellow No. 6); 9-(o-carboxyphen0yl)-6-hydroxy-2,4,5,7-tetraiodo-3H-xanthen-3-one, disodium salt, monohydrate (FD+C Red No. 3); 2-(1,3-dihydro-3-oxo-5-sulfo-2H-indol-2-ylidene)-2,3-dihydro-3-oxo-1H-indole-5-sulfonic acid disodium salt (FD+C Blue No. 2); and [phthalocyaninato (2-)]copper.

The composition may also optionally include at least one biological or therapeutic agent. The variety of biological/therapeutic agents that can be used in conjunction with the adhesive composition of the invention is vast. In general, biological/therapeutic agents which may be administered with adhesive/sealant compositions of the invention include, but are not limited to, anti-infective agents, such as antibiotics, antimicrobial agents (e.g. Diiodomethyl-p-tolylsulfone, 2,4,4′-Trichloro-2′-Hydroxydiphenyl Ether or combination thereof), antiseptics, bacteriocins, bacteriostats, disinfectants, fungicides, antibacterial, and antiviral agents; analgesics and analgesic combinations; anti-inflammatory agents; naturally derived or genetically engineered proteins, polysaccharides, glycoproteins, or lipoproteins; oligonucleotides, antibodies, antigens, cholinergics, cystostatics heparin neutralizers, procoagulants and hemostatic agents, such as prothrombin, thrombin, fibrinogen, fibrin, fibronectin, heparinase, Factor X/Xa, Factor VII/VIIa, Factor IX/IXa, Factor XI/XIa, Factor XII/XIIa, tissue factor, batroxobin, ancrod, ecarin, von Willebrand Factor, collagen, elastin, albumin, gelatin, platelet surface glycoproteins, vasopressin, vasopressin analogs, epinephrine, selectin, procoagulant venom, plasminogen activator inhibitor, platelet activating agents and synthetic peptides having hemostatic activity.

The composition may be used in surgical procedures as an adjunct to primary wound closure devices, such as staples, sutures, tapes, meshes to seal potential leaks of gasses, liquids, or solids. The surgical adhesive/sealant may be applied to tissue as a part of a surgical procedure in various forms, for example: liquid, powder, film, sponge or foam, impregnated fabric, impregnated sponge or foam, and spray. The instant adhesive compositions are particularly advantageous in a surgical context because of their absorbable nature.

The adhesive composition may be applied in single or multiple applications. For example, the adhesive compositions may be applied in a first layer, and after the first layer is allowed to fully or partially polymerize, a subsequent layer may be added. Such a process may be conducted numerous times, depending on the size of the wound and the amount of adhesive applied in each application.

In embodiments, the adhesive composition may be applied by any means known to those of skill in the art. By way of example, any suitable applicator may be used to apply the adhesive composite composition to a substrate.

Initiators, as described herein, may start polymerization of the polymerizable monomer composition. In these embodiments, maintaining the dual functional amine initiator and the polymerizable monomer composition separately is preferred

By way of example, where the polymerizable monomer or monomers are cyanoacrylate monomers, it is preferred that the cyanoacrylate monomer or monomers and the components associated with the cyanoacrylate monomer(s), such as inhibitors, plasticizers, preservatives and so on, as described, are kept separate from the dual functional amine until the time of use. By way of example, the polymerizable cyanoacrylate monomer or monomers and any additives such as plasticizer, inhibitor, preservative or other desired additive may form a polymerizable cyanoacrylate monomer composition which is kept separate or in a non-contacting relationship from the dual functional amine polymerization initiator until the time of use. At or just prior to the time the adhesive composition is to be used, the separate polymerizable monomer composition and the dual functional amine polymerization initiator are combined to form the adhesive composition.

Applicators which enable the separation of components until use and enable combination of two-component systems are well-known in the art. By way of example, the Applicator for CoSeal Sealant, distributed by Angiotech Pharmaceutical, may be used. In addition, the applicator disclosed in application Ser. No. 11/565,022, incorporated by reference herein, may also be used. By way of further example, a two-part syringe system may be used wherein the dual functional tertiary amine polymerization initiator is in one part and the polymerizable monomer composition is in another part. The components may be pushed together, combining at the time of use to form the adhesive composition which is dispersed for the desired application. Such a syringe system, for example, may utilize a T-shape configuration. Other two component systems are shown, for example, in U.S. Pat. Nos. 5,814,022 and 5,935,437.

In embodiments, a system for treating living tissue is provided with a first reservoir containing a biocompatible polymerizable monomer composition, a second reservoir in non-contacting relationship with the first reservoir containing an initiator and accelerator, and an applicator. The initiator preferably comprises the dual functional amine polymerization initiator. The biocompatible polymerizable monomer composition preferably comprises one or more cyanoacrylate monomer. The applicator is capable of combining the biocompatible polymerizable monomer composition and the dual functional amine polymerization initiator to form an adhesive composition and applying the adhesive composition to living tissue.

In embodiments, the initiator and accelerator may be placed in an applicator body in one container while the polymerizable cyanoacrylate monomer composition is stored in another container within the applicator body, so long as a non-contacting relationship between the polymerizable monomer composition and the dual functional amine polymerization initiator is maintained until use of the adhesive composition.

The adhesive composition may be packaged in any type of suitable container fabricated from materials including, but not limited to, glass, plastic, metal packages, and film-formed packages. Suitable containers preferably include those into which the compositions may be dispensed and sterilized without unacceptable damage to, or degradation of, the container or the components of the monomer composition. As disclosed in U.S. Patent Publication No. 2003/0039781, the entire disclosure of which is hereby incorporated by reference, post-halogenated (e.g., fluourinated) or silanized polymeric barrier layers on at least the monomer-contacting surfaces of the container provide a superior shelf-life for monomer compositions. Glass is especially preferred when sterilization is achieved with dry heat because of the lack of stability of many plastics at temperatures used for dry heat sterilization (typically at least about 140° C.). Examples of types of containers include, but are not limited to, ampoules, vials, syringes, pipettes, and the like.

The adhesive compositions described herein have multiple medical applications. For example, as an internal surgical adhesive and sealant, the adhesive can bond tissue to tissue, tissue to medical device (e.g. meshes, clips and films), and medical device to medical device. As a sealant, the composition can be coated on a tissue, on a medical device, or on the interface between a medical device and tissue to prevent leaks. The composition can be used to form films in situ that may have applications such as for the prevention of surgical adhesions. The composition can be used to form foams in situ that may have applications such as a filler (e.g. dead space removal, reconstructive, and cosmetic surgeries), bulking agents, tissue engineering (e.g. scaffolds) materials, and others where foams and sponges are useful. The composition can be formulated so that it is injectable and used to form gels in situ that are localized, and adherent to tissue, thus staying at the site where they are injected. The injectable formulation may have applications such as a delivery matrix for cells and other biologicals, bioactive agents and pharmaceutical or neutraceutical agents, as embolization agents, and as means to localize contrasting agents.

As a filler, the adhesive composition may be used as a facial, defect or void filler. For example, the composition may be applied in the interstices of an internal void and allowed to polymerize therein, such that the resultant polymer fills the internal cavities and voids, penetrating and conforming to the interstices and pores of the tissue. Thus, the composition may be used after a broad number of procedures having potential risk of dead space formation, including, but not limited to, radical mastectomy (i.e. breast and regional lymph nodes removal for cancer treatment), breast reconstruction and augmentation procedure, reconstructive or cosmetic abdominoplasty and liposuction, face-lift, cesarean section and hysterectomy in obese patients, orthopedic procedures on thigh region, incisional hernia repair, lipoma excision, and traumatic lesions, i.e. closed trauma.

### EXAMPLES

The present invention will be further understood by reference to the following non-limiting examples:

In the following examples two adhesive compositions (A and B) and a control adhesive composition (Control) are used to evaluate the dual functional amine initiator system in comparison with a blend of initiator and accelerator system. Compositions A and B are formulated to a viscosity of 200 to 250 centipoise. The control composition is approximately 40 to 55 centipoise. Table 1 details the general formulation of each composition. 2OCA indicates stabilized 2-octyl cyanoacrylate.

TABLE 1
Compositions Used for Testing

| | Approximate Weight percent (wt %) | | | | |
|---|---|---|---|---|---|
| Composition ID | 2OCA | Thickener | Plasticizer | Solvent | Dye |
| A | 83.8 | 8.7 | 6.3 | 1.2 | 0.004 |
| B | 79.2 | 9.5 | 6.3 | 5.0 | 0.004 |
| Control | 80.9 | 4.3 | 4.9 | 10.0 | 0.003 |

A glass ampoule filled with approximately 0.70 grams composition is prepared and sterilized via dry heat. The sterile ampoule is placed inside a flat-bottomed butyrate tube. The tube is sealed with a porous applicator tip. The porous applicator tip contained the initiator/accelerator system of interest. The initiator/accelerator system is applied to the porous applicator tip. The device may be sterilized using known methods such as ethylene oxide to render the final device sterile for application to living tissue.

In the following examples, the average exotherm is defined as the average of the maximum exotherms reached for a given set of data. The maximum exotherm is also reported for comparison.

### Example 1

Polymerization is attempted using composition B and the alkanolamine triisopropanolamine (TIPA) only. Three concentrations of TIPA were evaluated: 65, 130, and 240 microgram. The film did not set in a clinically acceptable timeframe. The test is abandoned if the film is not cured within five minutes. As a comparison, when using benzalkonium chloride (BAC) with the same composition, the film set in 118 seconds with an average exotherm of 50.1° C. and a maximum exotherm of 57.9° C. These devices are not terminally sterilized. Six units are tested.

### Example 2

Compositions A and B are polymerized using benzalkonium chloride (BAC) only. Three lots of composition A were evaluated: A1, A2, and A3. Three lots of composition B are also evaluated: B1, B2, and B3. One concentration of BAC is evaluated: 110 microgram (or 0.32 micromoles, using an average molecular weight of BAC as 340 g/mole;). The control composition is initiated using 40 microgram (or 0.12 micromoles, which correlates to 57 ppm when added to 0.7 g of cyanoacrylate) of BAC. These initiator levels allow a film to form in a clinically acceptable timeframe for the compositions under evaluation. Twelve devices are evaluated for average and maximum exotherm. In Table 2, the average and maximum exotherm are also reported as well as the decrease in both relative to the control composition.

TABLE 2
Higher Viscosity, Non-running 2-OCA Compositions A and B with
BAC alone and Compared with Control showing Average and
Maximum Exotherm and Decrease Relative to Control Composition
(BAC System: 110 microgram; Control: 40 microgram)

| | | | Average | Maximum |
|---|---|---|---|---|
| | Ave | Maximum | Temperature | Temperature |
| Composition | Temperature | Temperature | Decrease | Decrease |
| ID | (° C.) | (° C.) | (° C.) | (° C.) |
| A1 | 46 | 53 | 4 | 0 |
| A2 | 49 | 57 | 1 | −4 |
| A3 | 41 | 49 | 9 | 4 |
| B1 | 40 | 50 | 10 | 3 |
| B2 | 43 | 56 | 7 | −3 |
| B3 | 47 | 54 | 3 | −1 |
| Control | 50 | 53 | NA | NA |

The average setting temperature is lower for the test samples when compared to the control in all cases. The maximum setting temperatures in Table 2 in a given data set exceed that of the Control in 50% of the test data sets. A decrease in the average or maximum temperature as compared to Control is presented as a positive or desirable value, whereas an increase a negative or undesirable value.

The higher viscosity, non-running adhesive formulations, when cured with the single initiator, BAC, show maximum exotherms that exceed the control sample in 50% of the samples tested, while giving a cured film in less than 150 seconds.

### Example 3

### BAC and TIPA Blend Dual Initiator System Using the Impregnated Porous Tip:

A blend of TIPA and BAC is then evaluated for a dual accelerator/initiator system using the same method of Example 1 or 2. The cure data of setting times and setting temperatures are compiled in Table 3 with compositions A and B, each with two levels of BAC/TIPA compositions. Such a system demonstrate that the average and the maximum setting times were within clinically acceptable ranges. Also both the average setting temperature and the maximum setting temperature for the test devices are less than that of the Control, with one exception, and thus are mostly within the targeted range of desirable attributes.

TABLE 3
Setting Time and Setting Temperature for BAC/TIPA System with Higher Viscosity,
Non-running 2-OCA Compositions A and B

| | BAC/TIPA | BAC/TIPA | Average | Maximum | Average | Maximum |
|---|---|---|---|---|---|---|
| Composition | microgram,/ | micromoles/ | Time | Time | Temperature | Temperature |
| ID | microgram | micromoles | (seconds) | (seconds) | (degree C.) | (degree C.) |
| A1 (lot 1) | 78/1071 | 0.21/5.61 | 95 | 121 | 48 | 52 |
| B1 (lot 1) | 78/1071 | 0.21/5.61 | 76 | 98 | 54 | 60 |
| A1 (lot 1) | 114/1071 | 0.31/5.61 | 88 | 115 | 49 | 53 |
| B1 (lot 1) | 114/1071 | 0.31/5.61 | 76 | 113 | 48 | 52 |
| Control | 40/0 | 0.12/0 | 78 | 113 | 50 | 53 |

Additional data were generated at lower concentrations of BAC/TIPA. Here all the samples cured in a clinically acceptable timeframe. The cure temperatures are as shown in Table 4. A significant decrease in setting temperature as compared to control is clearly observed in Table 4.

TABLE 4
Setting Temperature with Several BAC/TIPA Compositions with Higher Viscosity,
Non-running 2-OCA Formulation A 1 and Compared with Control

| | BAC/ | | | | Average | Maximum |
|---|---|---|---|---|---|---|
| | TIPA | BAC/TIPA | Average | Maximum | Temp. | Temp. |
| Composition | microgram/ | micromoles/ | Temp. | Temp. | Decrease | Decrease |
| ID | microgram | micromoles | (C.) | (C.) | (C.) | (C.) |
| A1 (lot 1) | 55/907 | 0.15/4.75 | 41 | 46 | 9 | 7 |
| A1 (lot 1) | 45/829 | 0.12/4.34 | 41 | 45 | 9 | 8 |
| A1 (lot 1) | 45/706 | 0.12/3.70 | 39 | 43 | 11 | 10 |
| A1 (lot 1) | 35/706 | 0.10/3.70 | 37 | 42 | 13 | 11 |
| Control | 40/0 | 0.12/0 | 50 | 53 | N/a | N/a |

### Example 4

### Quadrol as a Dual Function Initiator System Using the Impregnated Porous Tip

An experimental investigation is undertaken to determine the suitability of Quadrol as an initiator in the porous tip for the same non-running topical skin adhesive composition. The setting time and temperature data are summarized in Table 5 for the same two 2-OCA formulations, A and B, each run with three concentrations of Quadrol as shown. The data in Table 5 indicates that Quadrol gives cure time in a clinically acceptable timeframe and the exotherm data are acceptable as compared to the Control. The higher concentrations of Quadrol have a tendency to give a faster cure and still maintain a desirable maximum setting temperature.

TABLE 5
Setting Time and Setting Temperature with Higher Viscosity, Non-running 2-OCA
Formulations A and B with Multiple Concentrations of Quadrol as Compared with Control

| | | Quadrol | Average | Maximum | Average | Maximum |
|---|---|---|---|---|---|---|
| Composition | Quadrol | Micro | Time | Time | Temperature | Temperature |
| ID | microgram | Moles | (seconds) | (seconds) | (degree C.) | (degree C.) |
| A 1 | 2762 | 9.46 | 86 | 114 | 46 | 50 |
| A 1 | 2071 | 7.09 | 100 | 119 | 45 | 49 |
| A 1 | 1381 | 4.73 | 105 | 145 | 47 | 52 |
| B 3 | 2762 | 9.46 | 102 | 141 | 43 | 44 |
| B 3 | 2071 | 7.09 | 102 | 112 | 43 | 45 |
| B 3 | 1381 | 4.73 | 121 | 202 | 43 | 45 |
| Control* | (BAC = 40) | (BAC = 0.12) | 78 | 113 | 50 | 53 |

What is claimed is:

1. An adhesive composition comprising:
a) one or more polymerizable cyanoacrylate monomers and
b) a multifunctional compound having at least two amine functional groups and at least one hydroxy moiety,
wherein each amine functional group has a different pKa value from the pKa value of the other amine functional group.

2. An adhesive composition according to claim 1 wherein the multifunctional compound has two amine functional groups that are tertiary amines in a basic environment.

3. An adhesive composition according to claim 1 wherein at least one of the amine functional groups exists in its quaternary ammonium form in a pH neutral environment and at least one other amine functional group is a tertiary amine in a pH neutral environment.

4. An adhesive composition according to claim 3 wherein the pKa value for one of the amine functional groups is at least 8 and the pKa value for the other amine functional group is less than 5.

5. An adhesive composition according to claim 3 wherein the pKa value for at least one of the amine functional groups is about 9.

6. An adhesive composition according to claim 5 wherein the pKa value for a second amine functional group is about 4.5.

7. An adhesive composition according to claim 1, wherein the multifunctional compound is represented by the following formula: 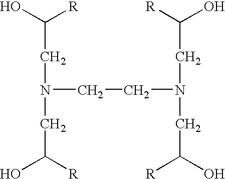
where each R group is independently H or C₁-C₃ alkyl.

8. An adhesive composition according to claim 1 wherein the multifunctional compound is represented by the formula:

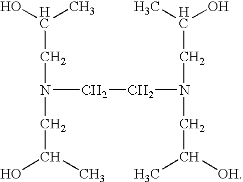

9. An adhesive composition according to claim 8 wherein the cyanoacrylate monomers are biocompatible.

10. An adhesive composition according to claim 9 wherein the cyanoacrylate monomers are bioabsorbable.

11. A system for treating living tissue comprising:
a) a first reservoir containing one or more polymerizable cyanoacrylate monomers, and
b) a second reservoir in a non-contacting relationship with the first reservoir containing a polymerization initiator for the one or more polymerizable cyanoacrylate monomers comprising a multifunctional compound having at least two amine functional groups and at least one hydroxy moiety,
wherein each amine functional group has a different pKa value from the pKa value of the other amine functional group; and
c) an applicator capable of combining the polymerizable cyanoacrylate monomer and the polymerization initiator to form an adhesive composition and then applying the adhesive composition to living tissue.

12. A system according to claim 11 wherein at least one of the amine functional groups exists in its quaternary ammonium form in a pH neutral environment and at least one other amine functional group is a tertiary amine in a pH neutral environment.

13. A system according to claim 11 wherein the pKa value for one of the amine functional groups is at least 8 and the pKa value for the other amine functional group is less than 5.

14. A system according to claim 11, wherein the multifunctional compound is represented by the following formula: 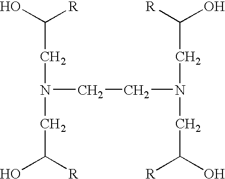
where each R group is independently H or C₁-C₃ alkyl.

15. A system according to claim 11 wherein the multifunctional compound is represented by the formula:

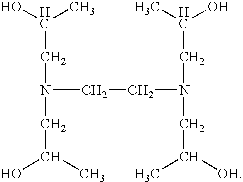

16. A system according to claim 15 wherein the polymerized cyanoacrylate monomers are biocompatible.

17. A system according to claim 17 wherein the polymerized cyanoacrylate monomers are bioabsorbable.

18. A method of treating living tissue, comprising applying to living tissue an adhesive composition according to claim 1.

19. A method for treating living tissue according to claim 18 wherein the adhesive composition is biocompatible and applied topically.

20. A method for treating living tissue according to claim 18 wherein the adhesive composition is bioabsorbable and applied internally in procedures selected from the group consisting of cardiovascular, peripheral-vascular, orthopedic, cardio-thoracic, gynecological, neuro- and general abdominal surgery.
